(19) United States
(12) Patent Application Publication
(10) Pub. No.: US 2023/0314434 A1
(43) Pub. Date: Oct. 5, 2023

(54) SEMI-QUANTITATIVE LATERAL FLOW DEVICES

(71) Applicant: Duke University, Durham, NC (US)

(72) Inventors: Ashutosh Chilkoti, Durham, NC (US); Cassio Fontes, Durham, NC (US); Angus Hucknall, Durham, NC (US)

(21) Appl. No.: 18/194,417

(22) Filed: Mar. 31, 2023

Related U.S. Application Data
(60) Provisional application No. 63/325,708, filed on Mar. 31, 2022.

(51) Int. Cl. G01N 33/569; G01N 33/543

(52) U.S. Cl. CPC G01N 33/56983; G01N 33/54388; G01N 33/54346

(57) ABSTRACT

Disclosed herein are lateral flow devices that can sensitively detect an analyte in a sample by using two different populations of nanoparticles. An example device comprises a porous substrate, the porous substrate comprising a sample zone, the sample zone including a detection nanoparticle and a control nanoparticle, wherein the detection nanoparticle and the control nanoparticle each include a different detection label; and a detection zone, the detection zone including a test line and a control line downstream from the test line. Also disclosed are methods and kits including the devices.

### CROSS-REFERENCE TO RELATED APPLICATIONS

This application claims priority to U.S. Provisional Patent Application No. 63/325,708 filed on Mar. 31, 2022, which is incorporated by reference herein in its entirety.

### STATEMENT REGARDING FEDERALLY SPONSORED RESEARCH

This invention was made with government support under grant number CBET2029361 awarded by the National Science Foundation; grant numbers P30-CA014236, R01-CA248491, and UH3-CA211232 awarded by the National Cancer Institute; grant number W81XWH-16-C-0219 awarded by the Department of Defense United States Special Operations Command; and grant number W81XWH-17-2-0045 awarded by the Combat Casualty Care Research Program (JPC-6). The government has certain rights in the invention.

### TECHNICAL FIELD

The present disclosure relates to semi-quantitative lateral flow devices and their use in methods of detecting analytes.

### INTRODUCTION

Lateral flow devices with improved sensitivity would be beneficial for the detection of numerous diseases.

### SUMMARY

In one aspect, disclosed are lateral flow devices comprising a porous substrate, the porous substrate comprising a sample zone, the sample zone including a detection nanoparticle and a control nanoparticle, wherein the detection nanoparticle and the control nanoparticle include a different detection label; and a detection zone, the detection zone including a test line and a control line downstream from the test line, wherein the test line includes a first capture agent and the control line includes a second capture agent, wherein the porous substrate defines a flow path through which a sample added to the sample zone flows under capillary action downstream from the sample zone into the detection zone.

In another aspect, disclosed are lateral flow devices comprising a porous substrate, the porous substrate comprising a sample zone, the sample zone including a detection nanoparticle and a control nanoparticle, wherein the detection nanoparticle includes a detection agent, a control molecule, and a detection label, wherein the control nanoparticle includes a control molecule, a detection label, and does not include a detection agent, wherein the detection nanoparticle is at least 3-fold greater in diameter than the control nanoparticle, and wherein the detection nanoparticle and the control nanoparticle include a different detection label; and a detection zone, the detection zone including a test line and a control line downstream from the test line, wherein the test line includes a first capture agent and the control line includes a second capture agent, wherein the porous substrate defines a flow path through which a sample added to the sample zone flows under capillary action downstream from the sample zone into the detection zone.

In another aspect, disclosed are lateral flow devices comprising a porous membrane mounted on a solid support, said porous membrane having a sample pad for receiving a sample comprising one or more target analytes at a first end, an absorbent pad at a second end, and a target area comprising one or more test lines positioned in between, said porous membrane permits capillary flow of the sample comprising the target analyte from the sample pad to the absorbent pad, in which the sample pad comprises at least two nanoparticles, each nanoparticle having a distinct color, and in which one or more of the nanoparticles is coated with an antibody directed against an analyte of interest and in which one or more nanoparticles comprise a control antibody or antibody fragment thereof, wherein the nanoparticles comprising the analyte of interest can bind to a test line in the presence of the antigen and wherein the nanoparticles comprising the control antibody can bind at a control line.

In another aspect, disclosed are methods of detecting an analyte, the method comprising contacting the sample zone of a disclosed lateral flow device with a sample; allowing the sample to laterally flow from the sample zone through the detection zone; and detecting the analyte.

In another aspect, disclosed are kits comprising a disclosed lateral flow device; and one or more packages, receptacles, labels, or instructions for use.

### BRIEF DESCRIPTION OF THE DRAWINGS

This patent or application file contains at least one drawing executed in color. Copies of this patent or patent application publication with color drawing(s) will be provided by the Office upon request and payment of the necessary fee.

FIG. 1 shows a schematic of an example lateral flow device.

FIG. 2 shows a schematic of an example lateral flow device corresponding to the varying levels of analyte detection.

FIG. 3 shows a photograph of an example lateral flow device.

FIG. 4 shows a photograph of a dose response of an antigen on an example lateral flow device ranging from highest amount (++++) to lowest amount (−).

FIG. 5 shows (i) a dose response of an antigen on an example lateral flow device ranging from highest amount (+++) to lowest amount (−), and (ii) test line sensitivity for an example device corresponding to a limit of detection (LOD) of 0.01 ng/mL.

FIG. 6 shows a dose response of an antigen on an example lateral flow device and corresponding concentration of the antigen.

FIG. 7 shows a zoomed in photograph from FIG. 6 visually depicting antigen concentration at 0.1 ng/mL and 0.01 ng/mL.

### DETAILED DESCRIPTION

The present disclosure is based, in part, on the development of a novel lateral flow device that incorporates a control line that changes color, shape, and/or position in response to the amount of an analyte present in a sample. The lateral flow device can also provide one or more secondary test lines (which can be hidden) that enable titration of the point at which a specific analyte load will cause a visual change in the control line. The secondary test lines can also be used for additional semi-quantitative discrimination of analyte status. For example, when the analyte is an antigen, the secondary test lines can be used for additional semi-quantitative discrimination of antigenemia status. Such readouts can be useful for providing additional information surrounding the time of infection, course of disease, prognosis and/or degree of contagiousness/likelihood of transmission, and the like.

### 1. Definitions

Unless otherwise defined, all technical and scientific terms used herein have the same meaning as commonly understood by one of ordinary skill in the art. In case of conflict, the present document, including definitions, will control. The materials, methods, and examples disclosed herein are illustrative only and not intended to be limiting. Methods and materials similar or equivalent to those described herein can be used in practice or testing of the disclosed invention. All publications, patent applications, patents and other references mentioned herein are incorporated by reference in their entirety.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “and” and “the” include plural references unless the context clearly dictates otherwise. The present disclosure also contemplates other embodiments “comprising,” “consisting of” and “consisting essentially of,” the embodiments or elements presented herein, whether explicitly set forth or not.

The modifier “about” used in connection with a quantity is inclusive of the stated value and has the meaning dictated by the context (for example, it includes at least the degree of error associated with the measurement of the particular quantity). The modifier “about” should also be considered as disclosing the range defined by the absolute values of the two endpoints. For example, the expression “from about 2 to about 4” also discloses the range “from 2 to 4.” The term “about” may refer to plus or minus 10% of the indicated number. For example, “about 10%” may indicate a range of 9% to 11%, and “about 1” may mean from 0.9-1.1. Other meanings of “about” may be apparent from the context, such as rounding off, so, for example “about 1” may also mean from 0.5 to 1.4.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are contemplated, and for the range 1.5-2, the numbers 1.5, 1.6, 1.7, 1.8, 1.9, and 2 are contemplated.

The term “antibody,” as used herein, refers to a protein including of one or more polypeptides substantially encoded by immunoglobulin genes or fragments of immunoglobulin genes. The recognized immunoglobulin genes include the kappa, lambda, alpha, gamma, delta, epsilon and mu constant region genes, as well as immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively. A typical immunoglobulin (antibody) structural unit is known to comprise a tetramer. Each tetramer is composed of two identical pairs of polypeptide chains, each pair having one “light” (about 25 kD) and one “heavy” chain (about 50-70 kD). The N-terminus of each chain defines a variable region of about 100 to 110 or more amino acids primarily responsible for antigen recognition. The terms variable light chain (V_{L}) and variable heavy chain (V_{H}) refer to these light and heavy chains, respectively. Antibody fragments include, but are not limited to, Fab, Fab′, Fab′-SH, F(ab′)₂, Fv, Fv′, Fd, Fd′, scFv, hsFv fragments, single-chain antibodies, cameloid antibodies, diabodies, and other fragments.

The term “antigen,” as used herein, refers to a molecule capable of being specifically bound by an antibody or a T cell receptor. The term “antigen” also encompasses T-cell epitopes. An antigen is additionally capable of being recognized by the immune system and/or being capable of inducing a humoral immune response and/or cellular immune response leading to the activation of B-lymphocytes and/or T-lymphocytes. In some embodiments, the antigen contains or is linked to a Th cell epitope. An antigen can have one or more epitopes (B-epitopes and T-epitopes). Antigens may include polypeptides, polynucleotides, carbohydrates, lipids, small molecules, polymers, polymer conjugates, and combinations thereof.

The term “biomarker,” as used herein, refers to a substance that is associated with a biological state or a biological process, such as a disease state or a diagnostic or prognostic indicator of a disease or disorder (e.g., an indicator identifying the likelihood of the existence or later development of a disease or disorder). The presence or absence of a biomarker, or the increase or decrease in the concentration of a biomarker, may be associated with and/or be indicative of a particular state or process. Biomarkers may include, but are not limited to, cells or cellular components (e.g., a viral cell, a bacterial cell, a fungal cell, a cancer cell, etc.), small molecules, lipids, carbohydrates, nucleic acids, peptides, proteins, enzymes, antigens and antibodies. A biomarker may be derived from an infectious agent, such as a bacterium, fungus or virus, or may be an endogenous molecule that is found in greater or lesser abundance in a subject suffering from a disease or disorder as compared to a healthy individual (e.g., an increase or decrease in expression of a gene or gene product).

The terms “biological sample” or “sample,” as used herein, refer to any material that is taken from its native or natural state, so as to facilitate any desirable manipulation or further processing and/or modification. A sample or a biological sample can include a cell, a tissue, a fluid (e.g., a biological fluid), a protein (e.g., antibody, enzyme, soluble protein, insoluble protein), a polynucleotide (e.g., RNA, DNA), a membrane preparation, and the like, that can optionally be further isolated and/or purified from its native or natural state. Example biological samples include, but are not limited to, blood, serum, plasma, lymph fluid, bile fluid, urine, saliva, mucus, sputum, tears, cerebrospinal fluid (CSF), bronchioalveolar lavage, nasopharyngeal lavage, rectal lavage, vaginal lavage, colonic lavage, nasal lavage, throat lavage, synovial fluid, semen, ascites fluid, pus, maternal milk, ear fluid, sweat, and amniotic fluid. A biological sample may be in its natural state or in a modified state by the addition of components such as reagents, or removal of one or more natural constituents (e.g., blood plasma). The biological sample may be pretreated as necessary by dilution in an appropriate buffer solution or concentrated, if desired. Any of a number of standard aqueous buffer solutions, employing one of a variety of buffers, such as phosphate, Tris, or the like, at physiological pH can be used.

The term “disease,” as used herein, refers to any abnormal condition and/or disorder of a structure or a function that affects a part of an organism. It may be caused by an external factor, such as an infectious disease, or by internal dysfunctions, such as cancer, cancer metastasis, and the like.

The term “downstream,” as used herein, indicates that the downstream location (e.g., on the lateral flow device) is further along the lateral flow device in the direction of fluid flow (capillary flow) than another location. Accordingly, if location B is downstream from location A, then fluid flowing through the lateral flow device will reach location A before reaching location B.

The term “limit of detection” or “LOD” is the point at which the measured value is larger than the uncertainty associated with its measurement. The LOD is defined as the lowest concentration at which a compound can be qualitatively identified, while quantification may not be accurate.

A “protein” or “polypeptide” is a linked sequence of 50 or more amino acids linked by peptide bonds. A peptide is a linked sequence of 2 to 50 amino acids linked by peptide bonds. The polypeptide and peptide can be natural, synthetic, or a modification or combination of natural and synthetic. Proteins and polypeptides include proteins such as binding proteins, receptors, and antibodies. The terms “polypeptide,” and “protein” are used interchangeably herein. “Primary structure” refers to the amino acid sequence of a particular peptide. “Secondary structure” refers to locally ordered, three dimensional structures within a polypeptide. These structures are commonly known as domains, e.g., enzymatic domains, extracellular domains, transmembrane domains, pore domains, and cytoplasmic tall domains, “Domains” are portions of a polypeptide that form a compact unit of the polypeptide and are typically 15 to 350 amino acids long. Example domains include domains with enzymatic activity or ligand binding activity. Typical domains are made up of sections of lesser organization such as stretches of beta-sheet and alpha-helices. “Tertiary structure” refers to the complete three-dimensional structure of a polypeptide monomer. “Quaternary structure” refers to the three-dimensional structure formed by the noncovalent association of independent tertiary units. A “motif” is a portion of a polypeptide sequence and includes at least two amino acids. A motif may be 2 to 20, 2 to 15, or 2 to 10 amino acids in length, in some embodiments, a motif includes 3, 4, 5, 6, or 7 sequential amino acids. A domain may be comprised of a series of motifs, which may be similar or different.

The term “small molecule,” as used herein, refers to inorganic or organic compounds having a molecular weight of less than 3,000 daltons.

The term “specifically binds,” as used herein, is generally meant that a molecule binds to a target molecule when it binds to that target molecule more readily than it would bind to a random, unrelated target.

The term “specific binding pair,” as used herein, refers to two molecules that exhibit specific binding to one another, or increased binding to one another relative to other molecules. A specific binding pair can exhibit functional binding activity such as, for example, a receptor and a ligand (such as a drug, protein, or carbohydrate), an antibody and an antigen, etc.; or structural binding activity such as, for example, protein/peptide and protein/peptide; protein/peptide and nucleic acid; and nucleotide and nucleotide etc. Examples of specific binding pairs include a detection agent and an analyte. The analyte can be a constituent of, or found in, a sample such as a biological fluid. Other examples of specific binding pairs include a control molecule and a second capture agent, and a complex formed between a detection nanoparticle and an analyte with a first capture agent. These examples are described in more detail below.

The term “subject” and “patient” are used interchangeably herein and refer to both human and nonhuman animals. The term “nonhuman animals” of the disclosure includes all vertebrates, e.g., mammals and non-mammals, such as nonhuman primates, rats, mice, rabbits, pigs, cows, sheep, goats, horses, dogs, cats, fish, birds, and the like. Typically, the subject is a mammal. A subject also refers to primates (e.g., humans, male or female; infant, adolescent, or adult) or non-human primates. In some embodiments, the subject is a primate. In some embodiments, the subject is a human.

The term “variant,” as used herein, refers to a peptide or protein that differs in amino acid sequence by the insertion, deletion, or conservative substitution of amino acids, but retain at least one biological activity relative to a reference peptide or protein. Representative examples of “biological activity” include the ability to be bound by a specific antibody or polypeptide or to promote an immune response. Variant can mean a substantially identical sequence. Variant can mean a functional fragment thereof. Variant can also mean multiple copies of a polypeptide. The multiple copies can be in tandem or separated by a linker. Variant can also mean a polypeptide with an amino acid sequence that is substantially identical to a referenced polypeptide with an amino acid sequence that retains at least one biological activity. A conservative substitution of an amino acid, i.e., replacing an amino acid with a different amino acid of similar properties (e.g., hydrophilicity, degree, and distribution of charged regions) is recognized in the art as typically involving a minor change. These minor changes can be identified, in part, by considering the hydropathic index of amino acids. See Kyte et al., J. Mol. Biol. 1982, 757, 105-132, which is incorporated by reference herein in its entirety. The hydropathic index of an amino acid is based on a consideration of its hydrophobicity and charge. It is known in the art that amino acids of similar hydropathic indexes can be substituted and retain protein function. In one aspect, amino acids having hydropathic indices of ±2 are substituted. The hydrophobicity of amino acids can also be used to reveal substitutions that would result in polypeptides retaining biological function. A consideration of the hydrophilicity of amino acids in the context of a polypeptide permits calculation of the greatest local average hydrophilicity of that polypeptide, a useful measure that has been reported to correlate well with antigenicity and immunogenicity, as discussed in U.S. Pat. No. 4,554,101, which is incorporated herein by reference. Substitution of amino acids having similar hydrophilicity values can result in polypeptides retaining biological activity, for example immunogenicity, as is understood in the art. Substitutions can be performed with amino acids having hydrophilicity values within ±2 of each other. Both the hydrophobicity index and the hydrophilicity value of amino acids are influenced by the particular side chain of that amino acid. Consistent with that observation, amino acid substitutions that are compatible with biological function are understood to depend on the relative similarity of the amino acids, and particularly the side chains of those amino acids, as revealed by the hydrophobicity, hydrophilicity, charge, size, and other properties.

A variant can be an amino acid sequence that is substantially identical over the full length of the amino acid sequence or fragment thereof. The amino acid sequence can be 80%, 81%, 82%, 83%, 84%, 85%, 86%, 87%, 88%, 89%, 90%, 91%, 92%, 93%, 94%, 95%, 96%, 97%, 98%, 99%, or 100% identical over the full length of the amino acid sequence or a fragment thereof.

The term “zone,” as used herein, refers to a defined area on the surface of a material (e.g., porous substrate). A zone may be identified and bounded by a distinct interface between two materials having different compositions.

### 2. Lateral Flow Devices

Disclosed herein are lateral flow devices (also referred to as “device,” “lateral flow assay,” “LFD,” and “LFA” herein) that can quickly and sensitively detect an analyte of interest via a semi-quantitative analysis. Lateral flow devices can be used for rapid detection of the presence or absence of a target analyte in a sample. In particular, “bar-code” or “ladder” lateral flow devices can provide a semi-quantitative measurement of the amount of analyte present in a sample, e.g., by providing multiple detection bands (e.g., in a detection zone) each of which can represent a different analyte concentration range. Due to their ease of use, lateral flow devices can be used for home testing, point of care testing, or laboratory applications.

The disclosed lateral flow devices include a porous substrate and a plurality of different zones, such as a sample zone and a detection zone. The lateral flow devices use distinct nanoparticle populations to semi-quantitatively measure the amount of analyte in a sample by visually detecting different band (or ladders), such as a test line and a control line. For example, the device can include a detection nanoparticle that can specifically bind to the analyte and a control nanoparticle that does not specifically bind to the analyte. The different nanoparticle populations can interact with the test line, the control line, or both. Upon contact with a biological sample, the detection nanoparticle can specifically bind to the analyte and can move along the device through capillary action where it can specifically bind to a test line(s), a control line, or combination thereof. In contrast, the control nanoparticle does not specially bind to the analyte and can move along the device through capillary action where it will not bind to a test line(s), but only to the control line. Depending on where and at what amount the different nanoparticle populations bind to the test line(s) and the control line, the user can visually determine the amount of analyte present in the sample.

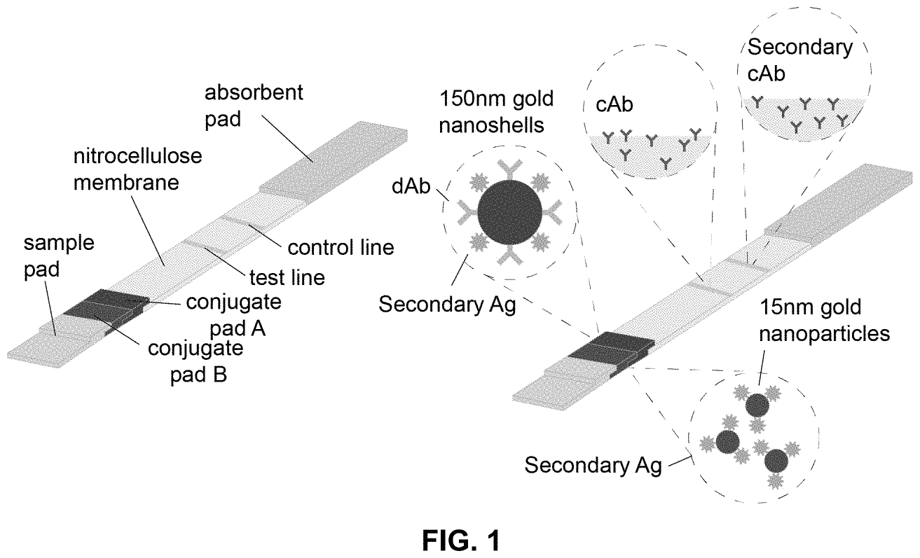

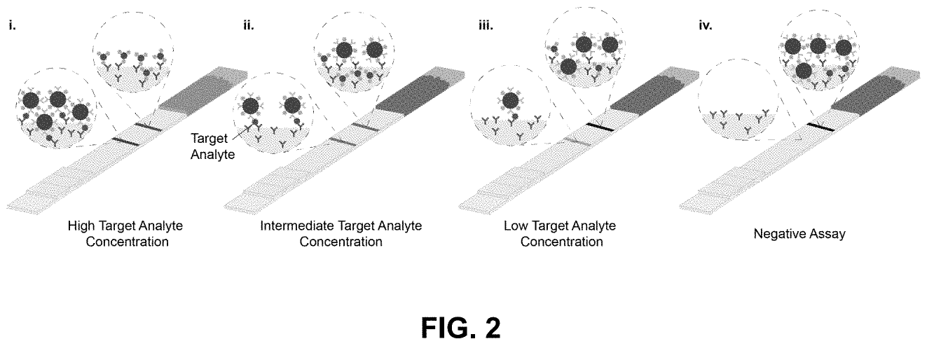

FIG. 1 illustrates an example lateral flow device. The example device includes a porous substrate (e.g., nitrocellulose membrane) including a sample pad, two conjugate pads (e.g., conjugate pad A and conjugate pad B), and an absorbent pad. The example device includes the absorbent pad at one end of the porous substrate, downstream from the sample pad and conjugate pads. The example device also includes a test line and a control line in between the conjugate pads and the absorbent pad. FIG. 1 further illustrates examples of the following: detection nanoparticle (e.g., 150 nm gold nanoshells); control nanoparticle (e.g., 15 nm gold nanoparticles); detection agent (e.g., detection antibody—dAb); control molecule (e.g., secondary antigen—Ag); first capture agent (e.g., capture antibody—cAb); and second capture agent (e.g., secondary capture antibody). FIG. 2 illustrates a dose response of an analyte for an example lateral flow device.

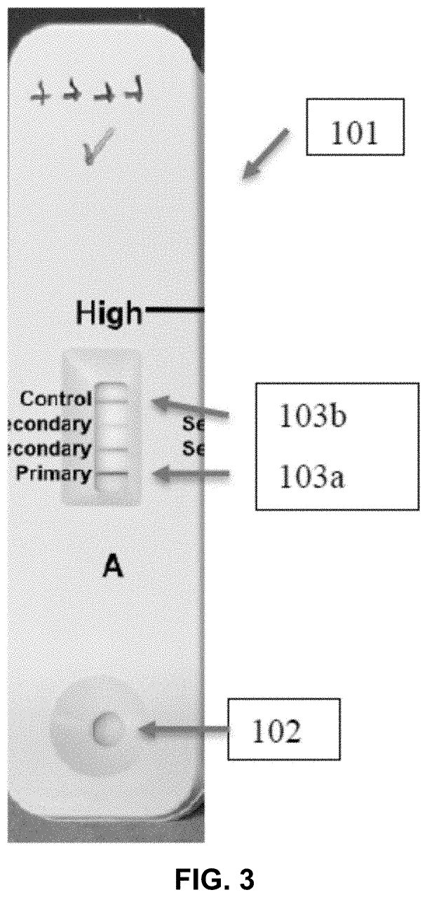

FIG. 3 shows an example lateral flow device (101) including a porous membrane mounted on a solid support, said porous membrane having a sample pad (102) for receiving a sample that can include one or more target analytes at a first end and an absorbent pad at a second end. The example device has a sample pad (102) for receiving a sample including one or more target analytes at a first end, an absorbent pad at a second end, and a target area including one or more test lines positioned in between. The porous membrane can permit capillary flow of the sample including the target analyte from the sample pad to a target area, in which the sample pad can include at least two different nanoparticles, each nanoparticle having a distinct color. The example device includes multiple nanoparticles. The nanoparticle(s) can be coated with an antibody directed against an analyte of interest and in which one or more nanoparticles can include a control antibody or antibody fragment thereof, wherein the nanoparticles including the analyte of interest can bind to a test line (103a) in the presence of analyte and wherein the nanoparticle(s) including the control antibody can bind at a control line (103b). The example device further includes a viewing window to observe the testing region.

In some embodiments, the lateral flow device includes a first nanoparticle and a second nanoparticle, each with a distinct color. In such embodiments, the first nanoparticle label can include the color green and can be coated with an antibody directed against an analyte of interest, in which the green nanoparticle label can bind to a test line in the presence of antigen, and also can bind to a control line with or without antigen present, and wherein the second nanoparticle label can be red and can be coated with a control antibody or antibody fragment in which this red nanoparticle label can bind only at the control line.

By using two distinct nanoparticle populations for detection of the analyte, the disclosed lateral flow devices can achieve advantageous sensitivity. For example, the device can have a lower limit of detection (LOD) of less than or equal to 1 ng/mL, less than or equal to 0.5 ng/mL, less than or equal to 0.25 ng/mL, less than or equal to 0.1 ng/mL, less than or equal to 0.09 ng/mL, less than or equal to 0.08 ng/mL, less than or equal to 0.07 ng/mL, less than or equal to 0.06 ng/mL, less than or equal to 0.05 ng/mL, less than or equal to 0.04 ng/mL, less than or equal to 0.03 ng/mL, less than or equal to 0.02 ng/mL, less than or equal to 0.01 ng/mL, or less than or equal to 0.009 ng/mL. In some embodiments, the device has a LOD of greater than or equal to 0.001 ng/mL, greater than or equal to 0.002 ng/mL, greater than or equal to 0.003 ng/mL, greater than or equal to 0.004 ng/mL, greater than or equal to 0.005 ng/mL, greater than or equal to 0.006 ng/mL, greater than or equal to 0.007 ng/mL, greater than or equal to 0.008 ng/mL, greater than or equal to 0.009 ng/mL, or greater than or equal to 0.01 ng/mL. In some embodiments, the device has a LOD of about 0.001 ng/mL to about 0.1 ng/mL, such as about 0.005 ng/mL to about 0.05 ng/mL, about 0.007 ng/mL to about 0.03 ng/mL, or about 0.009 ng/mL to about 0.02 ng/mL. In some embodiments, the device has a LOD of about 0.01 ng/mL. Because of the different nanoparticle populations, the LOD is analyte agnostic, as the device and nanoparticles thereof can be adapted to achieve the advantageous LOD for an analyte of interest.

A. Porous Substrates

The lateral flow device includes a porous substrate. The porous substrate includes a sample zone and a detection zone. The porous substrate can further include other zones as needed and depending on the application. The porous substrate can allow a mobile phase (e.g., a liquid sample) to flow through it by capillary action from the sample zone to the detection zone where a detectable signal, such as color changes or color differences on the device, may be generated to indicate the presence or absence of the target analyte. Accordingly, the porous substrate can define a flow path through which a sample added to the sample zone flows under capillary action downstream from the sample zone into the detection zone. As used herein, the term “capillary action” or “capillarity” refers to the process by which a molecule is drawn across the porous substrate due to such properties as surface tension and attraction between molecules.

The porous substrate can be made from any material sufficiently porous that can allow for capillary flow and detection of an analyte suitable for the disclosed devices. The porous substrate can include sintered glass or sintered ceramic, mineral, cellulose, fiberglass, nitrocellulose, polyvinylidene fluoride, nylon, charge modified nylon, polyethersulfone, or combination thereof. In some embodiments, the porous substrate includes sintered glass or sintered ceramic, mineral, cellulose, fiberglass, nitrocellulose, polyvinylidene fluoride, nylon, charge modified nylon, or polyethersulfone. In some embodiments, the porous substrate includes cellulose, fiberglass, or nitrocellulose. In some embodiments, the porous substrate includes nitrocellulose.

i. Sample Zone

The porous substrate includes a sample zone. The sample zone is configured to receive a sample. The sample once added to the sample zone can travel through the porous substrate toward the detection zone through capillary action. Accordingly, the detection zone is downstream from the sample zone.

In some embodiments, the sample zone includes a sample pad disposed on the porous substrate, in the porous substrate, or both. The sample pad may act as a filter that can remove debris, contaminants, and other unwanted substances present in the sample. The sample pad can include stored dried reagents (e.g., non-covalently bound) that can aid the detection of the analyte. For example, the dried reagents can adjust pH, ionic strength, and other properties of the sample fluid. Example materials that can be used for the sample pad include, but are not limited to, cellulose, nitrocellulose, fiberglass, cotton, woven or nonwoven paper, etc. Example reagents on the sample pad may include, but are not limited to, surfactants such as Triton X-100, Tween 20, or sodium dodecyl sulfate, etc.; polymers such as polyethylene glycol, poloxamer, polyvinylpyrrolidone (PVP), etc.; buffers such as phosphate-buffered saline, 4-(2-hydroxyethyl)-1-piperazineethanesulfonic acid (HEPES), Tris(hydroxymethyl) aminomethane (Tris), sodium borate, TRICINE, etc.; proteins such as albumin, etc.; enzymes such as protease, etc.; salts such as sodium chloride, sodium phosphate, sodium cholate, potassium phosphate, etc. The reagents can be applied to the sample pad by, e.g., soaking the material in the reagent solution, or through wicking the membrane via capillary flow. The treated sample pad can be dried by, e.g., air dry, heating at elevated temperatures, vacuum, or lyophilization.

The sample zone can also include at least two different populations of nanoparticles. For example, the sample zone can include a detection nanoparticle and a control nanoparticle. The different nanoparticles can be included in the sample pad, in a conjugate pad, or a combination thereof. In some embodiments, the detection nanoparticle corresponds to one conjugate pad and the control nanoparticle corresponds to a second conjugate pad different from the first conjugate pad. The conjugate pad(s) can be disposed on the porous substrate, in the porous substrate, or both. Example materials that can be used for the conjugate pads include, but are not limited to, cellulose, nitrocellulose, fiberglass, cotton, woven or nonwoven paper, etc. In some embodiments, the conjugate pad for the detection nanoparticle is downstream from the conjugate pad for the control nanoparticle. In some embodiments, the conjugate pad(s) are downstream from the sample pad. The detection nanoparticle and the control nanoparticle can be present in the sample pad and/or conjugate pad(s) as dried reagents (e.g., non-covalently bound). The nanoparticles can be applied to the sample pad and/or conjugate pad by, e.g., dipping the material in the reagent solution, or through spray coating the membrane. The treated sample pad and/or conjugate pad can be dried by, e.g., air dry, heating at elevated temperatures, vacuum, or lyophilization. Upon sample fluid reaching the sample pad and/or conjugate pad(s), the nanoparticles can be rehydrated. After rehydrating, the nanoparticles can interact with molecules of the sample and can travel along the porous substrate along with the fluid sample.

a. Detection Nanoparticles

The detection nanoparticle can include a detection agent, a control molecule, and a detection label. The detection agent is capable of specifically binding the analyte. Thus, the detection agent and the analyte can be a specific binding pair. The detection agent can be a peptide, a protein, a carbohydrate, a lipid, a small molecule ligand, a nucleic acid, or a combination thereof. In some embodiments, the detection agent includes a peptide, a protein, or a combination thereof. In some embodiments, the detection agent is an antibody or a fragment thereof. The detection agent can be adsorbed or chemically attached onto a surface of the detection nanoparticle. For example, in some embodiments, the detection agent is an antibody or a fragment thereof that is on a surface of the detection nanoparticle.

The control molecule allows the detection nanoparticle to specifically bind to the control line through, e.g., binding with a capture agent. Accordingly, the control molecule and the second capture agent can be a specific binding pair. The control molecule can be a peptide, a protein, a carbohydrate, a lipid, a small molecule ligand, a nucleic acid, or a combination thereof. In some embodiments, the control molecule includes a peptide, a protein, or a combination thereof. In some embodiments, the control molecule includes a peptide or a protein. In some embodiments, the control molecule is an antigen that the second capture agent is capable of specifically binding. The control molecule can be adsorbed or chemically attached onto a surface of the detection nanoparticle. For example, in some embodiments, the control molecule is a peptide or protein that is on a surface of the detection nanoparticle.

The detection label allows the detection nanoparticle to be detected and/or visualized directly or indirectly. The detectable label can include any composition detectable by spectroscopic, photochemical, biochemical, immunochemical, electrical, optical, or chemical means. Example detectable labels include, but are not limited to, fluorescent nanoparticles (e.g., quantum dots (Qdots)), metal nanoparticles, (e.g., gold nanoparticles) fluorescent dyes (e.g., fluorescein, texas red, rhodamine, green fluorescent protein, and the like, see, e.g., Molecular Probes, Eugene, Oreg., USA), radiolabels (e.g., ³H, ¹²⁵I, ³⁵S, ¹⁴C, ³²P, ⁹⁹TC, ²⁰³Pb, ⁶⁷Ga, ⁶⁸Ga, ⁷²As, ¹¹¹In, ^{113m}In, ⁹⁷Ru, ⁶²Cu, ⁵²Fe, ^{52m}Mn, ⁵¹cr, ¹⁸⁶Re, ¹⁸⁸Re, ⁷⁷As, ⁹⁰Y, ⁶⁷Cu, ¹⁶⁹Er, ¹²¹Sn, ¹²⁷Te, ¹⁴²pr, ¹⁴³pr, ¹⁹⁸Au, ¹⁹⁹Au, ¹⁶¹Tb, ¹⁰⁹pd, ¹⁶⁵Dy, ¹⁴⁹pm, ¹⁵¹pm ¹⁵³Sm, ¹⁵⁷Gd, ¹⁵⁹Gd, ¹⁶⁶Ho, ¹⁷²TM, ¹⁶⁹Y b, ¹⁷⁵Yb, ¹⁷⁷Lu, ¹⁰⁵Rh, ¹¹¹Ag, and the like), enzymes (e.g., horse radish peroxidase, alkaline phosphatase and others commonly used in an ELISA), various colorimetric labels, magnetic or paramagnetic labels (e.g., magnetic and/or paramagnetic nanoparticles), spin labels, radio-opaque labels, and the like.

In some embodiments, the detection label can be colorimetric, fluorescent, radioactive, magnetic, or enzymatic. In some embodiments, the detection label is colorimetric, fluorescent, or radioactive. In some embodiments, the detection label is colorimetric or fluorescent. In some embodiments, the detection label is colorimetric. In some embodiments, the detectable label is a gold nanoparticle or gold nanoshell that is detected through color. For example, the gold nanoparticle or gold nanoshell can have an absorbance wavelength of greater than or equal to 600 nm, greater than or equal to 625 nm, greater than or equal to 650 nm, greater than or equal to 675 nm, greater than or equal to 700 nm. Detectable color can be that of the visible spectrum. In some embodiments, the color is red, green, orange, purple, blue, or a combination thereof. In addition, the detection label can be adsorbed or chemically attached onto a surface of the detection nanoparticle, or the detection label can be a property of the material from which the nanoparticle is made.

The detection nanoparticle can include a number of different materials suitable for the disclosed devices. For example, the detection nanoparticle can include a gold nanoparticle, a gold nanoshell, a colloidal carbon, or a latex bead. In some embodiments, the detection nanoparticle includes a gold nanoparticle or a gold nanoshell. In some embodiments, the detection nanoparticle is a gold nanoshell. In some embodiments, the detection nanoparticle is a gold nanoparticle. In some embodiments, the detection nanoparticle is a gold nanoshell or gold nanoparticle having an absorbance wavelength of greater than or equal to 600 nm.

The detection nanoparticle can be included in the device at varying diameters. For example, the detection nanoparticle can have a diameter of about 100 nm to about 500 nm, such as about 110 nm to about 400 nm, about 115 nm to about 300 nm, about 125 nm to about 200 nm, about 100 nm to about 300 nm, or about 100 nm to about 200 nm. In some embodiments, the detection nanoparticle has a diameter of greater than or equal to 100 nm, greater than or equal to 110 nm, greater than or equal to 120 nm, greater than or equal to 130 nm, greater than or equal to 140 nm, greater than or equal to 150 nm, greater than or equal to 175 nm, or greater than or equal to 200 nm. In some embodiments, the detection nanoparticle has a diameter of less than or equal to 500 nm, less than or equal to 400 nm, less than or equal to 350 nm, less than or equal to 300 nm, less than or equal to 250 nm, less than or equal to 200 nm, less than or equal to 175 nm, or less than or equal to 150 nm. In some embodiments, the detection nanoparticle is about 150 nm.

It has been found that when the detection nanoparticle is significantly larger in diameter than the control nanoparticle, or the control nanoparticle is significantly smaller in diameter than the detection nanoparticle, the device can provide improved results. In some embodiments, the detection nanoparticle is at least 2-fold greater in diameter than the control nanoparticle, at least 2.5-fold greater in diameter than the control nanoparticle, at least 3-fold greater in diameter than the control nanoparticle, at least 3.5-fold greater in diameter than the control nanoparticle, at least 4-fold greater in diameter than the control nanoparticle, at least 4.5-fold greater in diameter than the control nanoparticle, or at least 5-fold greater in diameter than the control nanoparticle.

b. Control Nanoparticles

The control nanoparticle can include a control molecule and a detection label. In contrast to the detection nanoparticle, the control nanoparticle does not include a detection agent. The description regarding the control molecule and the detectable label above with respect to the detection nanoparticle can be applied to the control nanoparticle. However, the control nanoparticle and the detection nanoparticle include different detectable signals. For example, the control nanoparticle can be a gold nanoparticle that is visualized as red, and the detection nanoparticle can be a gold nanoparticle that is visualized as green. This difference in detectable signals can be used in numerous combinations as long as a difference in labels can be used to discern the analyte as disclosed herein. In some embodiments, the control nanoparticle and the detection nanoparticle include the same control molecule.

Similar to the detection nanoparticle, the control nanoparticle can include a number of different materials suitable for the disclosed devices. For example, the control nanoparticle can include a gold nanoparticle, a gold nanoshell, a colloidal carbon, or a latex bead. In some embodiments, the control nanoparticle includes a gold nanoparticle or a gold nanoshell. In some embodiments, the control nanoparticle is a gold nanoshell. In some embodiments, the control nanoparticle is a gold nanoparticle. In some embodiments, the control nanoparticle is a gold nanoparticle having an absorbance wavelength of less than or equal to 550 nm, such as less than or equal to 540 nm, less than or equal to 530 nm, less than or equal to 520 nm, less than or equal to 510 nm, less than or equal to 500 nm, or less than or equal to 450 nm. In some embodiments, the detection nanoparticle and the control nanoparticle include the same material. In some embodiments, the detection nanoparticle and the control nanoparticle include different materials. In some embodiments, the detection nanoparticle is a gold nanoshell and the control nanoparticle is a gold nanoparticle.

The control nanoparticle can be included in the device at varying diameters. For example, the control nanoparticle can have a diameter of about 5 nm to about 40 nm, such as about 10 nm to about 35 nm, about 10 nm to about 30 nm, about 5 nm to about 30 nm, about 5 nm to about 25 nm, or about 10 nm to about 20 nm. In some embodiments, the control nanoparticle has a diameter of greater than or equal to 5 nm, greater than or equal to 6 nm, greater than or equal to 7 nm, greater than or equal to 8 nm, greater than or equal to 9 nm, greater than or equal to 10 nm, greater than or equal to 15 nm, or greater than or equal to 20 nm. In some embodiments, the control nanoparticle has a diameter of less than or equal to 40 nm, less than or equal to 35 nm, less than or equal to 30 nm, less than or equal to 25 nm, less than or equal to 20 nm, less than or equal to 15 nm, or less than or equal to 10 nm. In some embodiments, the control nanoparticle is about 15 nm.

ii. Detection Zone

The porous substrate includes a detection zone. The detection zone is configured to detect a signal, e.g., from the different nanoparticles, from which the presence and/or amount of the analyte can be determined. The sample once added to the sample zone can travel through the porous substrate toward the detection zone through capillary action. From the sample zone to the detection zone, the sample fluid can resuspend the detection nanoparticle and the control nanoparticle, which can allow the detection nanoparticle to interact with the analyte if present. After the sample fluid has entered the detection zone, the different nanoparticles can interact with the test line(s) and the control line, which can allow for detection of signal denoting the presence and/or amount of the analyte.

The detection zone includes at least one test line and at least one control line. The test line and the control line can be used to accurately detect the presence of the analyte. In some embodiments, the detection zone includes a plurality of test lines. In some embodiments, the detection zone includes a plurality of test lines and a single control line. The detection zone can include 1 test line, 2 test lines, 3 test lines, 4 test lines, 5 test lines, 6 test lines, 7 test lines, 8 test lines, 9 test lines, 10 test lines, or more. Each test line can correspond to a concentration of the analyte. In some embodiments, the detection zone can include a first test line corresponding to a first concentration of an analyte, a second test line downstream from the first test line, wherein the second test line corresponds to a second concentration of the analyte, and an optional third test line downstream from the second test line, wherein the third test line corresponds to a third concentration. In some embodiments, the test lines correspond to a concentration of analyte that increases from upstream to downstream. Alternatively, the test lines can correspond to a concentration of analyte that decreases from upstream to downstream. The control line can be positioned in the detection zone downstream from the test line.

The test line(s) and control line can vary in distance in between each other. In some embodiments, the test line is located at least 1 mm, at least 2 mm, at least 3 mm, at least 4 mm, at least 5 mm, at least 6 mm, at least 7 mm, or more from another test line. In addition, the control line can be located (e.g., downstream) at least 1 mm, at least 2 mm, at least 3 mm, at least 4 mm, at least 5 mm, at least 6 mm, at least 7 mm, or more from the test line.

The test line and the control line include capture agents that allow specific interactions with the detection nanoparticle and the control nanoparticle. For example, the test line can include a first capture agent and the control line can include a second capture agent. The test line can include a capture agent that can specifically bind to a complex between the detection nanoparticle and the analyte. Accordingly, the first capture agent can be a specific binding partner for a complex of the detection nanoparticle and the analyte. In other words, the test line can detect the presence and/or amount of the analyte through a sandwich assay. The control line includes a capture agent that can specifically bind to the control molecule on the detection nanoparticle and/or the control nanoparticle. Thus, as mentioned above, the control molecule and the second capture agent can be a specific binding pair. And, consequently, in embodiments where there is no analyte in the sample, both detection nanoparticles and control nanoparticles can accumulate at the control line.

The capture agents can be a number of different molecules. For example, the first capture agent and the second capture agent can each individually include a peptide, a protein, a carbohydrate, a lipid, a small molecule ligand, a nucleic acid, or a combination thereof. In some embodiments, the first capture agent and the second capture agent are each individually a peptide or a protein. In some embodiments, the first capture agent and the second capture agent are each individually an antibody or fragment thereof.

In embodiments with a plurality of test lines, each test line can include the same capture agent or there can be some lines that include a different capture agent. For example, in an embodiment including 4 test lines, all four test lines can include the first capture agent, or, e.g., 2 of the 4 test lines can include the first capture agent and the other 2 test lines can include a third capture agent different from the first capture agent and the second capture agent.

B. Other Components

The device can include further components that can aid device performance. In some embodiments, the device further includes an absorbent pad downstream from the detection zone, and thus downstream from the control line. The absorbent pad can act as a sink, which can collect excess fluid and can prevent back-flow. Example materials that can be used for the absorbent pad include, but are not limited to, cellulose, nitrocellulose, fiberglass, cotton, woven, nonwoven paper, and the like—including combinations thereof.

In addition, the device can include a solid support. The solid support can be positioned under the porous substrate. Or in other words, the porous substrate can be positioned on the solid support. Example materials that can be used as the solid support include, but are not limited to, PVC, paper, plexiglass, other plastics, and combinations thereof.

### 3. Methods

Also disclosed herein are methods of detecting an analyte. The method can include contacting the sample zone of the disclosed device with a sample. A variety of different samples can be analyzed by the disclosed devices. In some embodiments, the sample includes blood, plasma, serum, saliva, or urine. Additionally, the analyte to be assessed is not generally limited. For example, the analyte can be a biomarker of an infectious agent (e.g., bacteria, virus, fungi, etc.); a cancer (e.g., breast cancer, colorectal cancer, colon cancer, lung cancer, prostate cancer, testicular cancer, brain cancer, etc.); a cardiovascular disease (e.g., coronary artery diseases (CAD) such as angina and myocardial infarction, stroke, hypertensive heart disease, rheumatic heart disease, cardiomyopathy, etc.); a metabolic disorder (obesity, type 2 diabetes mellitus, pancreatitis, dyslipidemia, nonalcoholic fatty liver disease, etc.); or an environmental agent (e.g., pesticides, toxins in general, esters of phthalmic acid, environmental DNA, marine biotoxins, etc.). The analyte may also be an antigen. In some embodiments, the analyte is an antigen derived from an infectious agent, a cancer, a cardiovascular disease, a metabolic disorder, or an environmental agent. In some embodiments, the analyte is an antigen derived from a bacterium, a fungus, a virus, or a parasite. In some embodiments, the analyte is an antigen derived from a virus. In some embodiments, the analyte is an antigen derived from a coronavirus. Example coronaviruses include, but are not limited to, 229E (alpha coronavirus), NL63 (alpha coronavirus), OC43 (beta coronavirus), HKU1 (beta coronavirus), MERS-CoV (beta coronavirus), SARS-CoV (beta coronavirus), and SARS-CoV-2. In some embodiments, the analyte is an antigen derived from SARS-CoV-2 or a variant thereof. After the sample contacts the sample zone, it can travel through the porous substrate via capillary action. While flowing through the sample zone, the detection nanoparticle can specifically bind to the analyte (if present), while the control nanoparticle does not bind the analyte.

The method can further include allowing the sample to laterally flow from the sample zone through the detection zone. Once in the detection zone, a complex formed between the detection nanoparticle and the analyte can specifically bind to the test line, e.g., through a specific interaction with the first capture agent. Detection nanoparticles that are not bound to analyte can specifically bind with the control line, e.g., through a specific interaction with the second capture agent. A complex between the detection nanoparticle and the analyte may also specifically bind to the control line if, e.g., the test line is saturated. In contrast, the control nanoparticle will only specifically bind to the control line, e.g., through specific interactions with the second capture agent. Accordingly, in some embodiments, the complex specifically binds to the test line, the control line, or both, and the control nanoparticle specifically binds only to the control line in the detection zone.

The method can also include detecting the analyte. Upon binding to the test line and/or control line, the detection nanoparticle can provide a visible test line and/or control line. And, upon binding to the control line, the control nanoparticle can provide a visible control line. The color of the control line can vary depending on the nanoparticle binding to it and amount thereof. In some embodiments, detection is done visually.

In some embodiments, the method includes using a disclosed lateral flow device including two nanoparticles, the method including first adding a sample fluid including the analyte of interest. Next, the sample fluid can be allowed to wick through a pad containing red and green nanoparticle labels and dissolve into the sample. The sample fluids can then be allowed to continue to transport through the device to the test region (e.g., detection zone), where the nanoparticle labels can bind to the test line only in the presence of the analyte. The sample fluid can continue to transport to additional test lines. These test lines can be used to control the amount of (green) nanoparticles that can reach the control line depending on the analyte concentration in the sample. The sample fluid transport can then continue through the target region to a control line where both (green and red) nanoparticle labels can bind to this control line. The color of the control line can be determined by the ratio of (red to green) nanoparticle labels remaining in the sample. Lastly, the results of the assay can be read.

In some embodiments, the following conditions are used as a two-color readout that correlates control line color to antigen levels: (1) When analyte (e.g., antigen) levels are low, only a small fraction of the (green) NP label can bind to the test lines, which can allow the majority of the (green) NP label to bind at the control line—this can result in a dark green/black control line when analyte (e.g., antigen) levels are low. (2) When analyte (e.g., antigen) levels are high, the vast majority of the (green) NP labels can bind to the test lines—this can result in primarily (red) NP labels binding to the control line, thereby resulting in a (red) control line when analyte (e.g., antigen) levels are high.

The description of the devices, porous substrates, sample zones, detection zones, detection nanoparticles, control nanoparticles, test lines, control lines, and other components can also be applied to the methods disclosed herein. 4. Kits

Also disclosed herein are kits that can be used for, e.g., detecting an analyte using the disclosed lateral flow devices. The kit can include a disclosed device, and one or more packages, receptacles, labels, or instructions for use. The kit may include at least one buffer. The kit may also include other components to facilitate using the device and methods thereof. Examples of such components include, but are not limited to, one or more additional reagents, such as one or more dilution buffers; one or more reconstitution solutions; one or more wash buffers; one or more storage buffers, one or more control reagents, (one or more additional component(s), such as a sample collection device (e.g., a syringe, cotton swab, tongue depressor, and the like), and the like. Components (e.g., reagents, components, etc.) may also be provided in a form that is usable in a particular assay, or in a form that requires addition of one or more other components before use (e.g. in concentrate or lyophilized form). Suitable buffers include, but are not limited to, phosphate buffered saline, sodium carbonate buffer, sodium bicarbonate buffer, borate buffer, Tris buffer, MOPS buffer, HEPES buffer, and combinations thereof. The choice of buffers and reagents will depend on the particular application, e.g., setting of the assay (point-of-care, research, clinical), analyte(s) to be assayed, the detection moiety used, etc. Such components may be provided individually or in combination(s) and may be provided in any suitable container such as a vial, a bottle, box, or a tube.

The kit may also include a packaging configured to contain the device and other components. The packaging may be a sealed packaging, such as a sterile sealed packaging. By “sterile” it is meant that there are substantially no microbes (such as fungi, bacteria, viruses, spore forms, etc.). In some embodiments, the packaging may be configured to be sealed, e.g., a water vapor-resistant packaging, optionally under an air-tight and/or vacuum seal.

Following construction of the device, it can be optionally dried, e.g., by mild desiccation, blow drying, lyophilization, or exposure to ambient air at ambient temperature, for a time sufficient for the article to be dry or at least macroscopically dry. Once the device is dry or at least macroscopically dry, it may be sealed in a container (e.g., such as an impermeable or semipermeable polymeric container) in which it can be stored and shipped to a user. Once sealed in a container, the device may have, in some embodiments, a shelf life of at least 2 to 4 months, or up to 6 months or more, when stored at a temperature of 25° C. (e.g., without loss of more than 20%, 30% or 50% of binding activity).

In addition to above-mentioned components, a subject kit can further include instructions for using the components of the kit to practice the disclosed methods. The instructions for practicing the methods are generally recorded on a suitable recording medium. For example, the instructions may be printed on a substrate, such as paper or plastic, etc. As such, the instructions may be present in the kits as a package insert, in the labeling of the container of the kit or components thereof (i.e., associated with the packaging or sub-packaging) etc. In some embodiments, the instructions are present as an electronic storage data file present on a suitable computer readable storage medium, e.g., CD-ROM, diskette, flash drive, etc. In some other embodiments, the actual instructions are not present in the kit, but means for obtaining the instructions from a remote source, e.g., via the internet, are provided. An example of this embodiment is a kit that includes a web address where the instructions can be viewed and/or from which the instructions can be downloaded. As with the instructions, this means for obtaining the instructions is recorded on a suitable substrate.

In some embodiments, the kit includes instructions that correlate the number, intensity, or both of visible test lines with the concentration of the analyte in the sample. In some embodiments, the kit includes a reference card that correlates the number and intensity of visible test lines with the concentrations of the analyte in the sample. In some embodiments, the kit further includes a smart device (e.g., tablet, smart phone, etc.) lateral flow device reader for the device(s).

The description of the devices, porous substrates, sample zones, detection zones, detection nanoparticles, control nanoparticles, test lines, control lines, and other components can also be applied to the kits disclosed herein.

The disclosed invention has multiple aspects, illustrated by the following non-limiting examples.

### 3. EXAMPLES

### Example 1

### Example Device Fabrication

Lateral flow devices can be fabricated with standard LFA manufacturing methods. An example lateral flow device was made as follows:

(1) Gold nanoshells (detection nanoparticles) were covalently conjugated to a detection reagent (antibody or antigen) and simultaneously to a control (antibody or antigen). Co-conjugation may not be needed, if detection reagent has an affinity tag or can have part of the antigen targeted by a control reagent that will go in the control line.

(2) Gold nanoparticles (control nanoparticles) were conjugated (covalently or not) to the control reagent. This control reagent may be an affinity tag, sequence or element that is part of the detection reagent, but it does not contain the sensing reagent that targets the analyte in the sample.

(3) Conjugate pads were washed with 0.05% tween in PBS and after air drying, were dipped or spray coated with the detection and/or control nanoparticles that have had their concentration adjusted and were supplemented with tween-20 to final 0.05% (v/v) and trehalose 10% (w/v) solution. Blocking reagents—buffers, surfactants, other proteins—may also be added to this solution.

(4) After functionalized with detection and control nanoparticle solutions, the conjugate pads were air or freeze-dried.

(5) In parallel, sample pads are treated with mixtures of buffers, surfactants and blocking reagents, that may include, PEG albumin, casein and/or specific reagents such as a high concentration of unrelated antibodies or animal serum, to mitigate cross-reactivity.

(6) Sample pads were air or freeze-dried.

(7) Nitrocellulose membranes (reaction matrix) were “impregnated” with control and test lines through contact or non-contact dispensing. Test lines include antibody or antigen that binds specifically to the target analyte in the sample of interest and forms a sandwich with the detection reagent in the detection nanoparticles.

(8) Control lines include antibody or antigen that binds to “secondary” target that was immobilized on the control nanoparticles and was either co-conjugated to the detection nanoparticles or is part of their sensing element.

(9) Once all elements are “fabricated”, the nitrocellulose membrane is assembled on a PVC card backing, absorbent membrane (wicking pad), followed by conjugate pads and finally the sample pad.

(10) Sheets of tests were then cut into (4 to 5 mm) individual strips and assembled in the individual use cassettes.

### Example 2

### Dose Response Antigen Run

In an example embodiment, the lateral flow assay (LFA) format relies upon the use of at least two (multiple labels can be used) different nanoparticle (NP) labels, each with a distinct color. The first NP label in one example is green and is coated with an antibody directed against an antigen of interest—this green NP label binds to a test line in the presence of antigen, and also binds to a control line with or without antigen present. The second NP label is red and is coated with a control antibody or antibody fragment—this red NP label binds only at the control line.

Other than the use of both red and green NP labels (most LFAs use only one color), the test proceeds in the same general format as most LFAs through the following steps: (1) Sample fluid is added. (2) Sample fluid is wicked through a pad containing red and green NP labels, allowing labels to dissolve into the sample. (3) Sample fluid transport continues through the device to a region containing a test line—green NP labels bind to this test line only in the presence of antigen. (4) Sample fluid transport continues to additional test lines. These test lines can be used to control the amount of green nanoparticles that will reach the control line depending on the antigen concentration in the sample. (5) Sample fluid transport continues through the device to a region containing a control line—both green and red NP labels bind to this control line. The color of the control line is determined by the ratio of red to green NP labels remaining in the sample.

The following conditions demonstrate how the two-color readout correlates to control line color: (1) When antigen levels are low, only a small fraction of the green NP label binds to the test lines, which allows the majority of the green NP label to bind at the control line—this results in a dark green/black control line when antigen levels are low. (2) When antigen levels are high, the vast majority of the green NP labels will bind to the test lines—this results in primarily red NP labels binding to the control line, thereby resulting in a red control line when antigen levels are high.

The secondary test lines define the antigen concentration at which green NP labels are capable of reaching the control line. This second test line is useful because, in order for the LFA to be as sensitive as possible, the green NP labels should be in excess when interacting with the first test line. Without the secondary test lines, this excess of green NP labels could result in green NP labels binding to the control line. However, through the use of secondary test lines, excess green NP labels can be bound prior to reaching the control line. This allows for a highly sensitive test where green NP labels are in excess at the first test line but are then fully bound by the secondary test lines. If necessary, these secondary test lines can be hidden from view by the LFA cartridge in order to avoid any confusion during assay interpretation. However, in addition to the color of the control line, additional semi-quantitative interpretation of the assay can occur through visual discrimination of the secondary test lines. This additional information from the secondary test lines is most useful when each secondary test line contains a smaller amount of capture probe (the capture probe is the printed element in each test line that allows for binding of the green NP labels in the presence of antigen). By utilizing secondary test lines with decreasing amounts of capture probe, the number of lines that appear green can be designed to correlate directly with antigen concentration where the additional appearance of secondary lines and/or darkening of secondary lines is indicative of a higher antigen concentration.

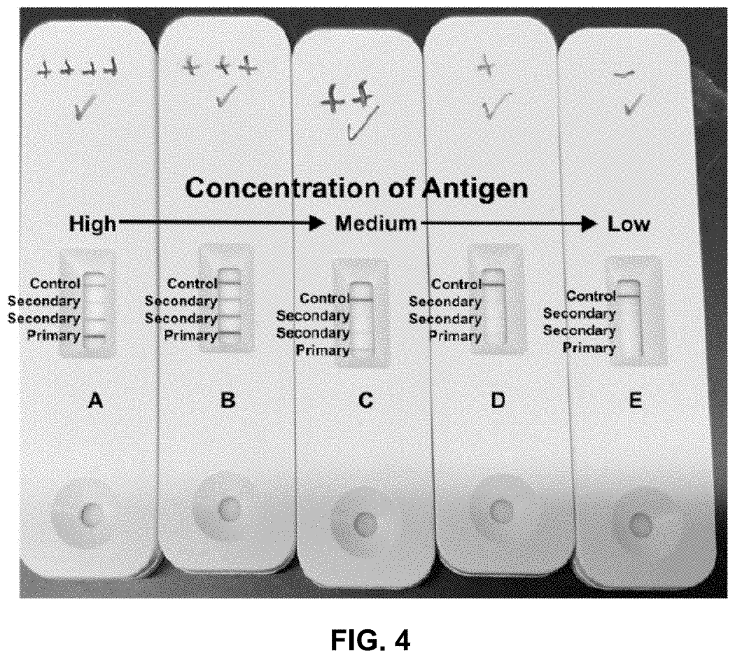

A dose response of antigen run on LFAs constructed in in accordance with one embodiment of the present disclosure is shown in FIG. 4. Antigen concentration decreases from left to right. The control line is visible at all concentrations, but at the high antigen concentration (A) the control line appears red, which is indicative of an extremely high antigen load— the appearance of this red control line can be designed to correlate with time of onset, severity, prognosis and/or contagiousness of disease. As antigen concentration drops, the primary and secondary test lines show decreased signal as follows: (1) At medium-high antigen concentration (B), both secondary lines are clearly visible. (2) At medium concentration (C), only one secondary line is clearly visible. (3) At medium low concentration (D), only primary line is visible. (4) At low or zero concentration (E), only the control line is visible as antigen concentration is below the LFA limit of detection.

### Example 3

### Multicolor LFA (McFLA): An Ultrasensitive Lateral Flow Assay

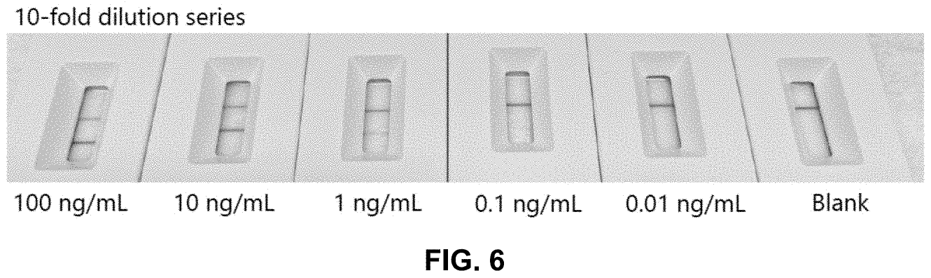

The use of a color changing control line to provide extra information on antigen concentration is shown in FIG. 6. The different lines can be tuned to desired concentration of antigen. In addition, the disclosed LFA is also compatible with standard LFA manufacturing methods. Generally, the method includes: (i) adding the sample to the sample pad of the device; (ii) allowing the sample fluid to wick through the sample pad which contains the one or more nanoparticles; (iii) allowing the labels to dissolve into the sample; (iv) allowing the sample to wick through the pad via capillary action to the test region (e.g., detection zone), wherein the nanoparticle comprising the analyte of interest will only bind to the test line if the analyte is present; (v) optionally allowing the sample to continue wicking through the test region to any additional test lines; and (vi) allowing the sample to wick through the test region to the control line; and (vii) reading the result.

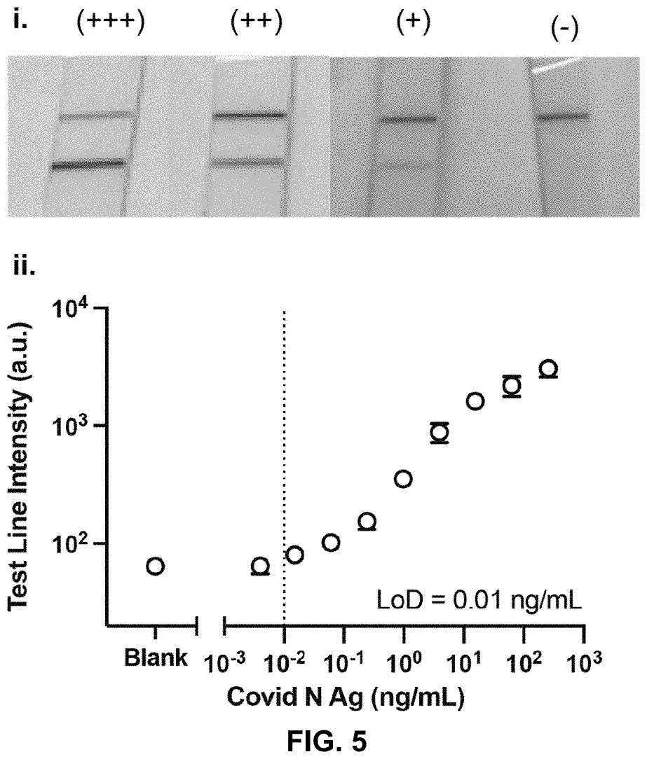

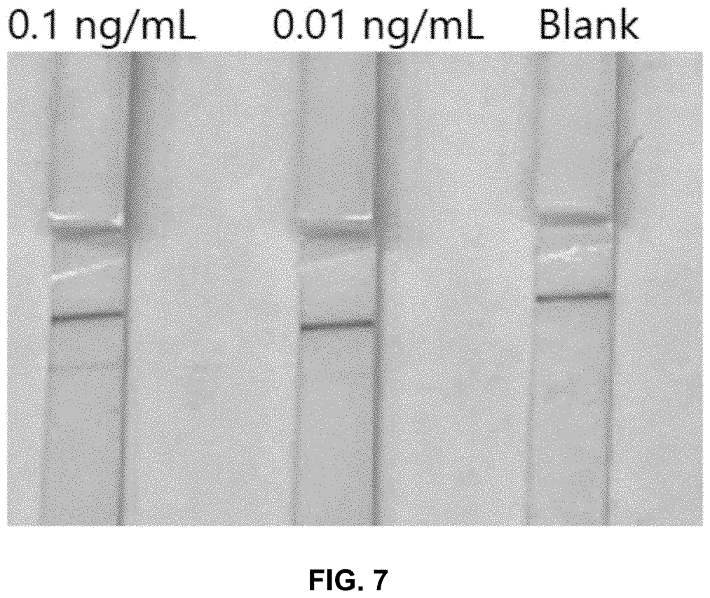

The disclosed LFA were used for the detection of SARS-CoV-2. As can be seen in FIG. 4, FIG. 5 and FIG. 6, the disclosed LFA detected SARS-CoV-2 antigen down to less than 0.1 ng/mL. Furthermore, the LFA showed a limit of detection (LOD) of about 0.01 ng/mL (FIG. 5 and FIG. 7), which outperformed other leading commercially available LFAs (Table 1).

Further description of the antibodies used for the examples can be found in Heggestad et al., Multiplexed, quantitative serological profiling of COVID-19 from blood by a point-of-care test; Science Advances, Vol. 7, No. 26, which is incorporated by reference herein in its entirety.

TABLE 1
Limit of Detection for Commercially Available LFAs

| Assay | Visual LoD |
|---|---|
| Abbott | 5-10 ng/mL |
| RapiGEN | 50-250 ng/mL |
| Coris BioConcept | 10-25 ng/mL |
| Roche SD | 2.5-5 ng/mL |

It is understood that the foregoing detailed description and accompanying examples are merely illustrative and are not to be taken as limitations upon the scope of the invention.

Various changes and modifications to the disclosed embodiments will be apparent to those skilled in the art. Such changes and modifications, including without limitation those relating to the chemical structures, substituents, derivatives, intermediates, syntheses, compositions, formulations, or methods of use of the invention, may be made without departing from the spirit and scope thereof.

For reasons of completeness, various aspects of the invention are set out in the following numbered clauses:

Clause 1. A lateral flow device comprising: a porous substrate, the porous substrate comprising a sample zone, the sample zone including a detection nanoparticle and a control nanoparticle, wherein the detection nanoparticle and the control nanoparticle each include a different detection label; and a detection zone, the detection zone including a test line and a control line downstream from the test line, wherein the test line includes a first capture agent and the control line includes a second capture agent, wherein the porous substrate defines a flow path through which a sample added to the sample zone flows under capillary action downstream from the sample zone into the detection zone.

Clause 2. The lateral flow device of clause 1, wherein the detection zone includes a plurality of test lines, each test line corresponding to a concentration of an analyte.

Clause 3. The lateral flow device of clause 1 or 2, wherein the detection zone includes: a first test line corresponding to a first concentration of an analyte, a second test line downstream from the first test line, wherein the second test line corresponds to a second concentration of the analyte, and an optional third test line downstream from the second test line, wherein the third test line corresponds to a third concentration.

Clause 4. The lateral flow device of any one of clauses 1-3, wherein the detection nanoparticle has a diameter of about 100 nm to about 500 nm.

Clause 5. The lateral flow device of any one of clauses 1-4, wherein the control nanoparticle has a diameter of about 5 nm to about 40 nm.

Clause 6. The lateral flow device of any one of clauses 1-5, wherein the detection nanoparticle and the control nanoparticle each individually comprise a gold nanoparticle, a gold nanoshell, a colloidal carbon, or a latex bead.

Clause 7. The lateral flow device of any one of clauses 1-6, wherein the detection nanoparticle comprises a detection agent, a control molecule, and a detection label.

Clause 8. The lateral flow device of any one of clauses 1-7, wherein the control nanoparticle comprises a control molecule and a detection label, and does not include a detection agent.

Clause 9. The lateral flow device of clause 7, wherein the detection agent comprises a peptide, a protein, a carbohydrate, a lipid, a small molecule ligand, a nucleic acid, or a combination thereof.

Clause 10. The lateral flow device of clause 9, wherein the detection agent is an antibody or a fragment thereof.

Clause 11. The lateral flow device of clause 7 or 8, wherein the control molecule is a peptide, a protein, a carbohydrate, a lipid, a small molecule ligand, a nucleic acid, or a combination thereof.

Clause 12. The lateral flow device of any one of clauses 1-11, wherein the detection label is colorimetric, fluorescent, radioactive, magnetic, or enzymatic.

Clause 13. The lateral flow device of any one of clauses 1-12, wherein the first capture agent and the second capture agent each individually comprise a peptide, a protein, a carbohydrate, a lipid, a small molecule ligand, a nucleic acid, or a combination thereof.

Clause 14. The lateral flow device of any one of clauses 1-13, wherein the first capture agent and the second capture agent are each individually an antibody or fragment thereof.

Clause 15. The lateral flow device of any one of clauses 1-14, wherein the first capture agent is capable of specifically binding a complex formed between the detection nanoparticle and an analyte.

Clause 16. The lateral flow device of any one of clauses 1-15, wherein the second capture agent is capable of specifically binding the control molecule.

Clause 17. The lateral flow device of any one of clauses 1-16, wherein the porous substrate is positioned on a solid support.

Clause 18. The lateral flow device of any one of clauses 1-17, wherein the porous substrate comprises an absorbent pad downstream from the detection zone.

Clause 19. The lateral flow device of any one of clauses 1-18, wherein the porous substrate comprises sintered glass or sintered ceramic, mineral, cellulose, fiberglass, nitrocellulose, polyvinylidene fluoride, nylon, charge modified nylon, polyethersulfone, or a combination thereof.

Clause 20. The lateral flow device of any one of clauses 1-19, wherein the device has a limit of detection (LOD) of less than or equal to 0.01 ng/mL.

Clause 21. A method of detecting an analyte, the method comprising: contacting the sample zone of the device of any one of clauses 1-20 with a sample; allowing the sample to laterally flow from the sample zone through the detection zone; and detecting the analyte.

Clause 22. The method of clause 21, wherein the sample includes blood, plasma, serum, saliva, or urine.

Clause 23. The method of clause 21 or 22, wherein the analyte is an antigen derived from an infectious agent, a cancer, a cardiovascular disease, a metabolic disorder, or an environmental agent.

Clause 24. The method of any one of clauses 21-23, wherein the analyte is an antigen derived from a bacterium, a fungus, a virus, or a parasite.

Clause 25. The method of any one of clauses 21-24, wherein the analyte is an antigen derived from SARS-CoV-2 or a variant thereof.

Clause 26. The method of any one of clauses 21-25, wherein the detection nanoparticle specifically binds the analyte to form a complex in the sample zone and the control nanoparticle does not bind the analyte.

Clause 27. The method of any one of clauses 12-26, wherein the complex specifically binds to the test line, the control line, or both, and the control nanoparticle specifically binds only to the control line in the detection zone.

Clause 28. The method of clause 27, wherein the complex specifically binding to the test line provides a visible test line, and the control nanoparticle specifically binding to the control line provides a visible control line.

Clause 29. The method of any one of clauses 21-28, wherein detecting the analyte is done visually.

Clause 30. A kit comprising: the lateral flow device of any one of clauses 1-20; and one or more packages, receptacles, labels, or instructions for use.

Clause 31. The kit of clause 30, further comprising instructions that correlate the number, intensity, or both of visible test lines with the concentration of the analyte in the sample.

What is claimed is:

1. A lateral flow device comprising:
a porous substrate, the porous substrate comprising
a sample zone, the sample zone including a detection nanoparticle and a control nanoparticle, wherein the detection nanoparticle and the control nanoparticle each include a different detection label; and
a detection zone, the detection zone including a test line and a control line downstream from the test line, wherein the test line includes a first capture agent and the control line includes a second capture agent,
wherein the porous substrate defines a flow path through which a sample added to the sample zone flows under capillary action downstream from the sample zone into the detection zone.

2. The lateral flow device of claim 1, wherein the detection zone includes a plurality of test lines, each test line corresponding to a concentration of an analyte.

3. The lateral flow device of claim 2, wherein the detection zone includes:
a first test line corresponding to a first concentration of an analyte,
a second test line downstream from the first test line, wherein the second test line corresponds to a second concentration of the analyte, and
an optional third test line downstream from the second test line, wherein the third test line corresponds to a third concentration.

4. The lateral flow device of claim 1, wherein the detection nanoparticle has a diameter of about 100 nm to about 500 nm.

5. The lateral flow device of claim 1, wherein the control nanoparticle has a diameter of about 5 nm to about 40 nm.

6. The lateral flow device of claim 1, wherein the detection nanoparticle and the control nanoparticle each individually comprise a gold nanoparticle, a gold nanoshell, a colloidal carbon, or a latex bead.

7. The lateral flow device of claim 1, wherein the detection nanoparticle comprises a detection agent, a control molecule, and a detection label.

8. The lateral flow device of claim 1, wherein the control nanoparticle comprises a control molecule and a detection label, and does not include a detection agent.

9. The lateral flow device of claim 7, wherein the detection agent comprises a peptide, a protein, a carbohydrate, a lipid, a small molecule ligand, a nucleic acid, or a combination thereof.

10. The lateral flow device of claim 9, wherein the detection agent is an antibody or a fragment thereof.

11. The lateral flow device of claim 7, wherein the control molecule is a peptide, a protein, a carbohydrate, a lipid, a small molecule ligand, a nucleic acid, or a combination thereof.

12. The lateral flow device of claim 1, wherein the detection label is colorimetric, fluorescent, radioactive, magnetic, or enzymatic.

13. The lateral flow device of claim 1, wherein the first capture agent and the second capture agent each individually comprise a peptide, a protein, a carbohydrate, a lipid, a small molecule ligand, a nucleic acid, or a combination thereof.

14. The lateral flow device of claim 13, wherein the first capture agent and the second capture agent are each individually an antibody or fragment thereof.

15. The lateral flow device of claim 1, wherein the first capture agent is capable of specifically binding a complex formed between the detection nanoparticle and an analyte.

16. The lateral flow device of claim 1, wherein the second capture agent is capable of specifically binding the control molecule.

17. The lateral flow device of claim 1, wherein the porous substrate is positioned on a solid support.

18. The lateral flow device of claim 1, wherein the porous substrate comprises an absorbent pad downstream from the detection zone.

19. The lateral flow device of claim 1, wherein the porous substrate comprises sintered glass or sintered ceramic, mineral, cellulose, fiberglass, nitrocellulose, polyvinylidene fluoride, nylon, charge modified nylon, polyethersulfone, or a combination thereof.

20. The lateral flow device of claim 1, wherein the device has a limit of detection (LOD) of less than or equal to 0.01 ng/mL.

21. A method of detecting an analyte, the method comprising:
contacting the sample zone of the device of claim 1 with a sample;
allowing the sample to laterally flow from the sample zone through the detection zone; and
detecting the analyte.

22. The method of claim 21, wherein the sample includes blood, plasma, serum, saliva, or urine.

23. The method of claim 21, wherein the analyte is an antigen derived from an infectious agent, a cancer, a cardiovascular disease, a metabolic disorder, or an environmental agent.

24. The method of claim 21, wherein the analyte is an antigen derived from a bacterium, a fungus, a virus, or a parasite.

25. The method of claim 21, wherein the analyte is an antigen derived from SARS-CoV-2 or a variant thereof.

26. The method of claim 21, wherein the detection nanoparticle specifically binds the analyte to form a complex in the sample zone and the control nanoparticle does not bind the analyte.

27. The method of claim 26, wherein the complex specifically binds to the test line, the control line, or both, and the control nanoparticle specifically binds only to the control line in the detection zone.

28. The method of claim 27, wherein the complex specifically binding to the test line provides a visible test line, and the control nanoparticle specifically binding to the control line provides a visible control line.

29. The method of claim 21, wherein detecting the analyte is done visually.

30. A kit comprising:
the lateral flow device of claim 1; and
one or more packages, receptacles, labels, or instructions for use.

31. The kit of claim 30, further comprising instructions that correlate the number, intensity, or both of visible test lines with the concentration of the analyte in the sample.
